(19) United States
(12) Patent Application Publication
(10) Pub. No.: US 2018/0256043 A1
(43) Pub. Date: Sep. 13, 2018

(54) METHODS AND DEVICES USING PHOTOPLETHYSMOGRAPHY IN THE OPTIMIZATION OF CARDIOPULMONARY RESUSCITATION

(71) Applicant: Xhale, Inc., Gainesville, FL (US)

(72) Inventor: Richard J. Melker, Newberry, FL (US)

(21) Appl. No.: 15/537,023

(22) PCT Filed: Dec. 17, 2015
(86) PCT No.: PCT/US15/66449

§ 371 Date: Jun. 16, 2017

Related U.S. Application Data
(60) Provisional application No. 62/092,890, filed on Dec. 17, 2014.

(51) Int. Cl. A61B 5/0295; A61H 31/00

(52) U.S. Cl. CPC A61B 5/0295; A61H 31/005; A61H 31/007; A61H 2201/5097; A61H 2201/5043; A61H 2230/207; A61H 2230/40; A61H 2230/04; A61H 2201/5092; A61H 2230/25

(57) ABSTRACT

Provided according to embodiments of the invention are methods of improving the effectiveness of chest compressions as part of cardiopulmonary resuscitation (CPR). Such methods include monitoring PPG signals from a PPG sensor secured to a nose during at least one chest compression and increasing, decreasing or maintaining a at least one of the depth, duration and frequency of at least one subsequent chest compression based on a waveform parameter of the PPG signals. Related devices and systems are also provided.

### CROSS-REFERENCE TO RELATED APPLICATIONS

This application claims the benefit of U.S. Provisional Application No. 62/092,890, filed Dec. 17, 2014, the contents of which are incorporated herein by reference in their entirety.

### FIELD OF THE INVENTION

The present invention relates to biological sensors, and in particular, to the use of biological sensors to monitor physiological parameters of patients. The present invention also relates to cardiopulmonary resuscitation.

### BACKGROUND OF THE INVENTION

Photoplethysmography, or “PPG”, is an optical technique for detecting blood volume changes in a tissue. In this technique, one or more emitters are used to direct light at a tissue and one or more detectors are used to detect the light that is transmitted through the tissue (“transmissive PPG”) or reflected by the tissue (“reflectance PPG”). The volume of blood, or perfusion, of the tissue affects the amount of light that is transmitted or reflected. Thus, the PPG signal varies with changes in the perfusion of the tissue.

The blood volume in a tissue changes with each heartbeat, and so the PPG signal also varies with each heartbeat. Traditionally, this component of the PPG signal is referred to as the “AC component” component of the signal, and is also often referred to as the “pulsatile component.” Blood volume is also affected by other physiological processes in the body, including respiration, venous blood volume, sympathetic and parasympathetic tone and certain pathologies. The changes in the PPG signal due to these and other physiological processes, along with changes in the PPG signal due to noise caused by non-physiological processes such as ambient light and bodily movement, have traditionally been referred to collectively as the “DC component.”

Pulse oximetry is a well-known physiological monitoring tool whereby PPG is used to monitor arterial blood oxygen saturation (SpO₂) in an individual. In typical pulse oximetry, red and IR radiation is transmitted through a tissue of the individual. The amplitude of the AC component of the PPG signal for IR and red wavelengths is sensitive to changes in SpO₂ due to the difference in the light absorbance of oxygenated and deoxygenated hemoglobin at these wavelengths. From their amplitude ratio, using the DC components to normalize their respective signals, the SpO₂ can be estimated. Traditionally, pulse oximetry has been performed at peripheral sites, but in recent years, alternative monitoring sites have been investigated, including at the nose (e.g., septum, nasal alar), ear (e.g., earlobe, concha), and forehead.

The nose, in particular the nasal alar, has recently been identified by the present inventor as a particularly promising site for PPG. Traditional sites for monitoring PPG, such as fingers and toes, generally provide a relatively small PPG signal, and the quality of this signal may be negatively impacted by sympathetic innervation in these tissue sites. Thus, in some cases, pulse oximetry measurements at peripheral sites may be unavailable or unreliable. Furthermore, due to the small signal size, the DC component signal from traditional peripheral sites may not be of sufficient strength and quality to allow for the DC signal to be used to reliably monitor physiological processes.

The nasal alar region has been shown by the inventor to provide a very large PPG signal relative to other sites of the body, including the fingers, toes and ears, and a relatively high quality signal due to its lack of sympathetic innervation. The improved PPG signal at the nasal alar site has allowed for a number of physiological parameters, including respiration rate, blood flow, respiratory effort and venous capacitance to be effectively extracted from the DC signal. The improved PPG signal is likely due to the unique dual blood supply to the nose. The nasal alae (and the nasal septum) are supplied by the last branch of the external carotid artery (via the facial artery) and the first branch of the internal carotid artery (via the ophthalmic artery) with rich anastomoses especially in the area of the alae and septum (sometimes referred to as Kiesselbach's plexus). These vessels are relatively immune to clinical conditions that diminish the PPG signal at other sites (e.g. cold, anxiety, hypoperfusion). Examples of patents and applications that describe the use the nasal alar site to obtain PPG signals, as well as a description of parameters and physiological processes that may be extracted from such signals, include U.S. Pat. Nos. 6,909,912, 7,127,278, 7,024,235, 7,785,262, 7,887,502, 8,161,971, 8,679,028 and 8,641,635, the entire contents of each of which are incorporated herein by reference in their entirety.

### SUMMARY OF EMBODIMENTS OF THE INVENITON

Provided according to embodiments of the present invention are methods for improving the effectiveness of chest compressions as part of cardiopulmonary resuscitation (CPR). In some embodiments, such methods include monitoring PPG signals from a PPG sensor secured to the nose of an individual during at least one chest compression; and increasing, decreasing or maintaining a depth, duration or frequency of at least one subsequent chest compression based on a waveform parameter of the PPG signals. In some cases, the PPG sensor is secured to a nasal alar or nasal septum of the individual. The PPG signals may also be used to determine when a return to spontaneous circulation (ROSC) occurs and how long to continue compressions after ROSC.

Any suitable waveform parameter may be monitored in the methods described herein. For example, the waveform parameter may include an amplitude or an area under the curve for the PPG waveform. In some embodiments, the waveform parameters are determined based on an isolated component signal of the PPG waveform. In certain embodiments, PPG signals are separated into at least one of an AC component signal stream and a DC component signal stream, and the depth, duration or frequency of the at least one additional chest compression is maintained, increased or decreased based on a waveform parameter of the AC component signal, the DC component signal, or both. Furthermore, in some embodiments, the depth, duration or frequency of the chest compression may be maintained, increased or decreased based on the waveform parameter and an additional physiological parameter such as blood oxygen saturation or a respiratory parameter.

Also provided according to embodiments of the invention are automated chest compression devices. In some embodiments, such devices include a compressor for performing chest compressions on an individual; and a controller for activating the compressor to perform chest compressions on the individual, wherein the controller receives and processes PPG signals from a PPG sensor secured to the nose; and wherein the controller calculates a waveform parameter of the PPG signals during a chest compression and directs the compressor to maintain, increase or decrease at least one of a depth, duration or frequency of at least one subsequent chest compression based on the waveform parameter.

### BRIEF DESCRIPTION OF THE DRAWINGS

FIG. 1 shows a PPG waveform that may be used to calculate waveform parameters according to some embodiments of the invention.

FIG. 2 provides a graphic illustrating how methods according to embodiments of the invention may be used iteratively to maintain, improve or optimize the efficacy of chest compressions in CPR.

FIGS. 3a-3c provide an example of a graphical displays that may be used to guide a caregiver in optimizing CPR according to embodiments of the invention.

FIG. 4 shows a simplified depiction of an automated chest compression device according to an embodiment of the invention.

### DETAILED DESCRIPTION OF EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers refer to like elements throughout the specification. It will be understood that, although the terms first, second, etc. may be used herein to describe various elements, these elements should not be limited by these terms. These terms are only used to distinguish one element from another. Thus, a first element discussed below could be termed a second element without departing from the teachings of the present invention.

As discussed above, the nose (e.g., nasal alae and nasal septum) has a large PPG signal relative to other sites of the body, and a relatively high quality signal due to its lack of sympathetic innervation. In addition, based on the proximity of the brain, the blood flow at the nose is proportional to cerebral blood flow, and so measurement of blood flow or blood volume at the nose can be used as a surrogate for blood flow to the brain. A goal of CPR is to provide blood flow to the brain and heart with sufficient flow for defibrillation to be effective. Thus, the PPG signal at the nose may provide a suitable means for determining how effective each chest compression is at providing cerebral blood. By using the PPG signal as a guide, the at least one of depth, duration and frequency of chest compressions can be modified, improved, and potentially optimized, thus improving the outcome and efficacy of CPR.

As such, provided according to embodiments of the invention are methods for improving the effectiveness of chest compressions as part of cardiopulmonary resuscitation (CPR). In some embodiments, such methods include one or more of the following: securing a photoplethysmography (PPG) sensor onto a nose of an individual; monitoring PPG signals from the PPG sensor during at least one chest compression; and increasing or decreasing at least one of the depth, duration and frequency of at least one subsequent chest compression based on a waveform parameter of the PPG signals during the at least one chest compression.

### The Individual

As used herein, an individual, also referred to as a patient, includes any mammal, including humans of any age. The individual may be monitored in any care setting including, but not limited to, hospitals (e.g., operating room (OR), intensive care unit (ICU), general care floors, or during transport therein); nursing homes, medical offices, medical transport, and homes.

### The PPG Sensor

The PPG sensor may be secured to any suitable portion of the nose. However, in particular embodiments, the PPG sensor is secured to the nasal alar or nasal septum of the individual. The term “secure” means to attach sufficiently to the skin to allow for a suitable PPG signal to be generated. In some cases, the sensor body is configured to secure onto the skin such that no additional support is necessary to allow for a suitable PPG signal to be reliably generated. However, in some cases, the sensor may be secured with the aid of an external support, for example, an additional structural support, a wire or cord, or an adhesive product such as tape. Such supports may be desirable to stabilize the sensor to prevent against signal loss, for example, due to the patient's movement (e.g., shivering), or due to movement (e.g., jostling, pulling, pushing) of the sensor or a cable attached thereto.

The PPG sensors include one or more components that emit light, and such components will be referred to herein as “emitters.” As used herein, the term “light” is used generically to refer to electromagnetic radiation, and so the term includes, for example, visible, infrared and ultraviolet radiation. Any suitable type of emitter may be used, but in some embodiments, the emitter is a light-emitting diode (LED). In particular embodiments, a first emitter emits light at a first wavelength, and a second emitter emits light at a second wavelength. In some cases, a single emitter may emit light at a first wavelength and a second wavelength. One or more photodetectors, also referred to as “detectors”, are also included in the PPG sensor. The detector is configured to detect light from an emitter, and this detected light generates a PPG signal. Any suitable photodetector may be used. However, examples of photodetectors include photodiodes, photoresistors, phototransistors, light to digital converters, and the like.

The PPG sensor measures arterial pulsatile blood volume and flow (beat to beat blood volume x heart rate/minute) and so will detect the change in beat to beat arterial blood volume directly when chest compressions are applied to the individual. This is distinguished from tissue or cerebral oximetry (near infrared spectroscopy [NIRS]/rSO₂) sensors/technology that can only measure average oxygen saturation (venous weighed in a ratio of 3:1 venous to arterial oxygenation) and microvascular flow values over many beats/compressions with the selected tissue bed. Thus, NIRS technology measures microvascular tissue perfusion while pulse oximetry measures arterial blood volume/flow. The PPG sensors secured to the nose will specifically measure the arterial blood volume perfusing the brain (with chest compressions), and so will provide a meaningful guide to determining the correct depth, duration and/or frequency of chest compressions.

While any suitable nasal PPG sensor may be used, non-limiting examples of nasal PPG sensors that may be used are described in U.S. Pat. Nos. 8,755,857, 8,641,635 and 8,679,028, and in U.S. Publication No. 2014/0005557, the relevant contents of each of which are hereby incorporated by reference.

### The PPG Signals & Waveform Parameters

The PPG sensor transmits raw PPG signals to a signal processing device. As used herein, the term “PPG signals” refers to the PPG waveform (signal over time), and may also be called the PPG signal stream. In some cases, the raw PPG signals are “conditioned” or filtered before being monitored or used in parameter calculations. In general, such conditioning is achieved by band pass filters, which may filter out undesirably high or low frequency noise in the signal. As used herein, a “raw PPG signal” includes both completely unprocessed signals and those that have been conditioned. While in some embodiments, the PPG signal from only one wavelength of light is processed and/or monitored, in other embodiments, PPG signals may be obtained from two or more wavelengths of light (e.g., IR and red wavelengths) and in some cases, comparing waveform parameters at different wavelengths may allow for more accurate determination of the effectiveness of chest compressions.

In some embodiments of the invention, a raw PPG signal is used to assess the efficacy of chest compressions. However, in other embodiments of the invention, the raw PPG signals are separated into an isolated AC component signal stream (the pulsatile component), an isolated DC component signal stream (the non-pulsatile component), or both, and the isolated AC and/or DC signal streams, optionally with the raw PPG signal, may be used to monitor the effectiveness of chest compressions. The isolated AC component may be particularly useful in evaluating the changes in arterial blood volume with a particular depth, duration and/or frequency of chest compression. The separation of the AC and DC component signal streams may be achieved by a number of different methods, but in some embodiments, the components are separated as discussed in U.S. Pat. No. 8,529,459, which is herein incorporated by reference in its entirety. As another example, in some embodiments, the DC component signal stream is determined by interpolating the peaks of the raw signal stream and interpolating the troughs of the raw signal stream and then averaging the two interpolated lines (interpolated peak line and interpolated trough line) to produce an isolated DC component signal stream (the DC component waveform). The isolated DC component stream may be subtracted from the raw signal stream to produce the AC component signal stream. Other methods of separating the pulsatile from the non-pulsatile components of PPG signals that are known in the art may also be used.

While in some embodiments, only the AC or the DC component signal stream is monitored to determine the effectiveness of the CPR, in some embodiments, both the AC and the DC component signal streams are monitored. One reason for monitoring both the AC and DC component signal streams is that the AC and DC components both may provide information regarding blood flow and the strength of each signal may vary based on the position or physiological condition of the individual.

A number of waveform parameters may be obtained from the raw or isolated PPG signals, and such parameters may be used to assess the effectiveness of one or more chest compressions, and to determine an appropriate depth, duration and/or frequency of subsequent chest compressions. For example, in some embodiments, the amplitude of one or more of the raw signal, the AC component and the DC component may be monitored to determine the effectiveness of the chest compression(s). The amplitude of the PPG signal at the nose is typically proportional to the blood flow to the nose, and thus, proportional to blood flow to the brain. Thus, monitoring the amplitude of one or more PPG signals over time may be useful to assess whether a particular depth of compression is providing sufficient or optimal blood to the brain. Another waveform parameter that may be monitored is the area under the curve. The area under the curve for one or more of the raw PPG signal, the isolated AC component and the isolated DC component may be monitored to determine the effectiveness of the chest compression(s). Any of the standard methods for determining area under a curve may be used. The area under the curve may be particularly valuable for assessing the appropriate duration of the chest compression, whereby if a longer duration increases the area under the curve, more blood may be directed to the brain and the CPR may be considered more effective. Conversely, if the area under the curve does not increase with longer chest compressions or even diminishes, then it may be considered that a shorter duration of chest compressions is sufficient or desirable.

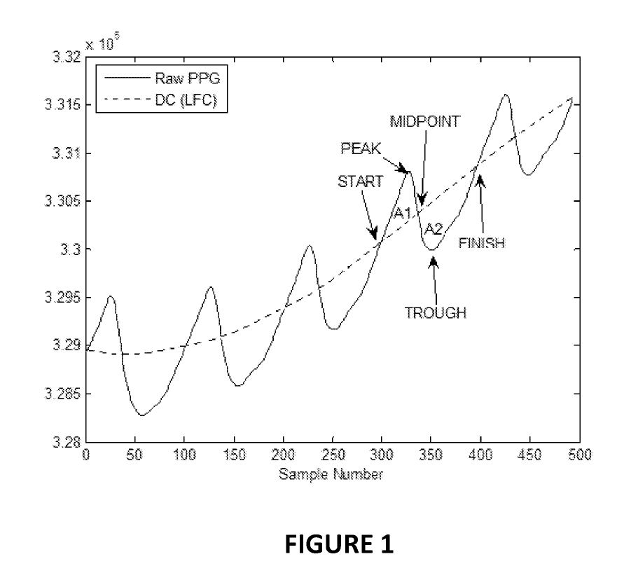

Any other suitable waveform parameter may be used to monitoring the effectiveness of the chest compressions. However, in some embodiments, the DC and raw PPG signal may be evaluated together to define the waveform parameters described below and in FIG. 1:
The Top Area is the area under the curve of the positive portion (portion above the DC component) of the cardiac signal. It is labeled as A1 in FIG. 1.
The Bottom Area is the area under the curve of the negative portion (portion under the DC component) of the cardiac signal. It is labeled as A2 in FIG. 1.
The Peak Trough Ratio is the Peak Height divided the Trough Depth. The Area Ratio is the Top Area (A1 in FIG. 1) divided by the Bottom Area (A2 in FIG. 1).
The Mean DC is the mean DC amplitude taken from “START” to “FINISH” for each heart beat.
The Min DC is the maximum DC amplitude taken from “START” to “FINISH” for each heart beat.
The Max DC is the minimum DC amplitude taken from “START” to “FINISH” for each heart beat.
The DC Swing is the difference in amplitude between the Max DC and the Min DC.
The DC Area is the area under the curve between the DC signal and a calculated “baseline” DC. The calculated baseline is the average DC values over a prior predefined time period.

### Monitoring the PPG Signals & Waveform Parameters

The PPG signals may be monitored in any suitable fashion, but are typically monitored with a signal processing device, such as a general-purpose microprocessor, a digital signal processor (DSP) or application specific integrated circuit (ASIC). The singular term “signal processing device” may include two or more individual signal processing devices. Such signal processing devices may be adapted to execute software, which may include an operating system and one or more applications, as part of performing the functions described herein. In electronic communication with the signal processing device may be a computer memory, such as a read-only memory (ROM), random access memory (RAM), and the like. Any suitable computer-readable media may be used in the system for data storage. Computer-readable media are capable of storing information that can be interpreted by microprocessor. This information may be data or may take the form of computer-executable instructions, such as software applications, that cause the signal processing device to perform certain functions and/or computer-implemented methods. Depending on the embodiment, such computer-readable media may include computer storage media and communication media.

Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In particular embodiments of the invention, a signal processing device is configured to isolate the AC component signal stream, the DC component signal stream, or both, from a raw PPG signal. The device may use any suitable processing method to achieve this, including the methods described above. In some embodiments, the microprocessing device conditions the PPG signals prior to separation of the AC and DC component signal streams. This signal processing device for processing the PPG signals may be the same or a different signal processing device as that used to monitor the PPG signals.

According to some embodiments of the invention, waveform parameters from the PPG signals during one, two or more chest compressions are monitored/calculated. In some embodiments, if the waveform parameter meets preselected criteria, a person or device may increase at least one of the depth, duration and frequency of subsequent chest compressions. For example, if the amplitude or other waveform parameter does not increase (or decreases) with the one or more chest compression(s), the person or device may increase the depth of chest compression, may extend the duration of the chest compression, or may increase the frequency of chest compressions (reduce the time between chest compressions). Alternatively, if a waveform parameter meets a different set of preselected criteria, the person or device may decrease the depth of chest compression, may shorten the duration of the chest compression, or may slow down the rate of chest compressions (more time between subsequent chest compressions). The person or device may try each modification individually (e.g, change depth, but not duration or frequency) or may increase or decrease two or more modalities at the same time (e.g., change depth and duration at one time).

If a waveform parameter meets a different set of preselected criteria, the person or device may maintain at least one of depth, duration and frequency of chest compression. A signal processing device may instruct a caregiver to vary at least one of depth, duration and frequency of chest compression as described above. Preselected criteria may include, but are not limited to, a certain amplitude or amplitude range (or other waveform criteria range), a certain percentage (e.g., 5%, 10%, 15%, 20% or greater) or absolute increase or decrease over baseline of a former value for the amplitude or other waveform parameter, and a trend (e.g., positive slope change) in the waveform parameter over time.

In some cases, the waveform parameters may be monitored in combination with other physiological signals to determine whether to maintain, increase or decrease the at least one of the depth, duration and frequency of the chest compression. In some cases, at least one of the depth, duration and frequency may be determined based both on the waveform parameters and on parameters derived from other uses of the PPG signal (e.g., blood pressure, SpO₂, respiratory parameters, etc.). In other cases, at least one of the depth, duration and frequency of compression may be determined based both on the waveform parameters and physiological parameters determined from other sensors (e.g., ECG or other heart rate monitors, or respiratory monitors such as thermistor). Thus, in some embodiments, the preselected criteria may include, along with the criteria based on the waveform parameters, criteria based on signals obtained from other physiological sensors and/or criteria based on other parameters from the PPG signal. Examples of such preselected criteria include, but are not limited to, the oxygen saturation, the blood pressure of the individual, and the increase or decrease in electrical activity in the heart.

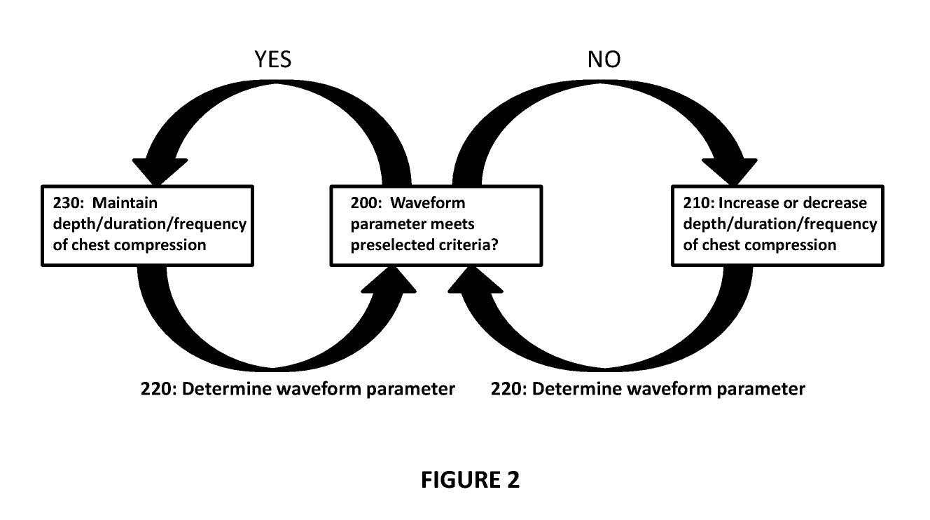

The process of monitoring the effectiveness of the depth and/or duration of compression described above may be iterated. For example, referring to FIG. 2, during or after one or more chest compression(s), the waveform parameter(s) calculated from a PPG sensor at the nose may be evaluated and assessed for meeting the preselected criteria 200. Then, if the preselected criteria are not met, at least one of the depth, duration and frequency of compression may be increased or decreased 210. The PPG signals obtained during or after this chest compression may be evaluated for the waveform parameter 220 and again assessed for meeting the preselected criteria 200. If the preselected criteria are not met, the at least one of depth, duration and frequency of compression may be increased or decreased 210 again, and it may proceed in an iterative fashion until the preselected criteria are met. If the waveform parameter is evaluated 200 and preselected criteria are met, then the at least one of depth, duration and frequency of compression may be maintained 230. However, the PPG signals and waveform parameter may continue to be determined 220 and evaluated 200 to ensure that the preselected criteria continue to be met. In some cases, the preselected criteria may change over time, e.g., if a first preselected criteria is met, and the at least one of depth, duration and frequency is maintained, and then a second preselected criteria may be used to determine whether maintenance of the at least one of depth, duration and frequency is appropriate over time.

Additionally, the determination of whether to increase or decrease the depth and/or duration of a chest compression may not happen after every chest compression. In some case, the PPG signals during two or more chest compressions may be evaluated by the signal processing device, and in some cases, the average or trend in the waveform parameter may be used to assess whether the preselected criteria are met.

In some embodiments of the invention, the waveform parameters of the PPG signals are calculated on a substantially real time basis (e.g, less than every 1, 2, 3, 4 or 5 seconds), so that information regarding the effectiveness of each chest compression may be conveyed to the caregiver to guide the depth and/or duration of subsequent chest compressions. Furthermore, in some embodiments, the PPG signals may provide information regarding the effectiveness of a chest compression even when the individual is taking (or administered) certain medications such as vasopressors which cause peripheral vasoconstriction. Therefore, the at least one of depth, duration and frequency of chest compressions may be monitored regardless of the medications taken by the individual.

### The Chest Compressions

The waveform parameter is determined and compared with preselected criteria, and in response, the at least one of the depth, duration and frequency of the chest compressions is maintained, increased or decreased. As used herein, the depth is the distance the patient's chest is downwardly compressed (e.g., 1.0 or 1.5 to 2 inches), the duration is how long the chest is compressed (e.g., 50% of a cardiac cycle) and the frequency is the number of chest compressions in a particular time period (e.g., 100/minute). The chest compressions may be performed manually, and instruction to a caregiver may be provided via the signal processing device. For example, a graphic on a monitor may increase in size or area when the waveform parameter is increasing, and decrease when the waveform parameter decreases. Alternatively or additionally, numerical values or written instructions may be displayed to guide the caregiver in order to increase, improve, or optimize the effectiveness of the chest compressions.

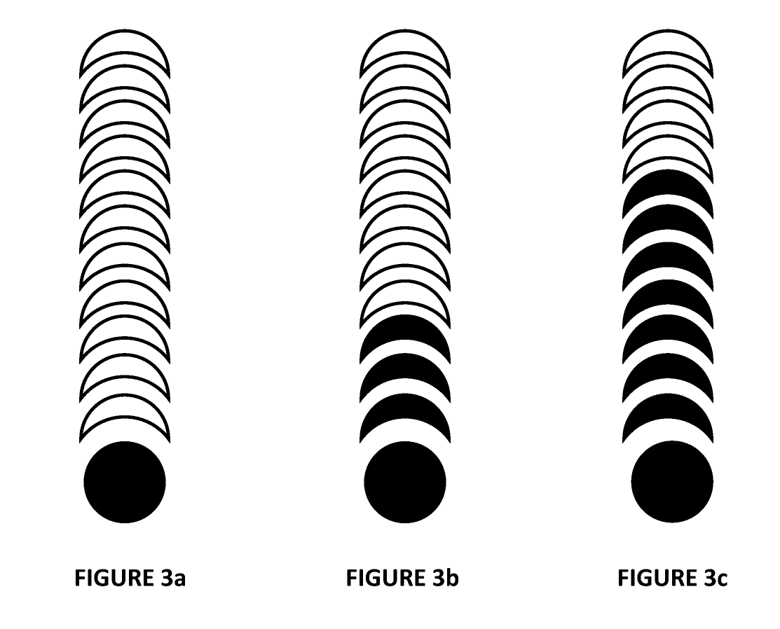

As an example, referring to FIGS. 3a-3c, when chest compressions are not improving blood volume to the brain as evidenced by the PPG signal/waveform parameter, a graphic may show low or no illumination in a graphical display (FIG. 3a). As the waveform parameter increases (effectiveness of CPR increases), the graphical display may provide an indication that the blood volume to the brain is increasing, in this case, by an illumination of additional bars (area) in the graphical display (FIG. 3b). As blood volume to the brain further increases, additional bars (area of the graphical display) may be illuminated (FIG. 3c). If an increase in depth or duration of a chest compression decreases blood flow, then in some cases, the illuminated area of the graphical display may decrease, such as back to the level shown in FIG. 3b. This graphical display may guide a medical professional/caregiver to improve the effectiveness of the chest compressions.

The graphical display may be arranged in a variety of different configurations, and any suitable configuration may be used, including different sizes and shapes of bars or areas of the graphical display, and including 2D or 3D configurations. Any suitable type of emitter may be used in the graphical array as well, but in some embodiments, the emitter is a light-emitting diode (LED), and in some embodiments, a red-green-blue (RGB) LED. Examples of other types of emitters/display types include Liquid Crystal Displays (LCD), Organic LEDs (OLED), Plasma Displays (PD) and Cathode Ray Tube (CRT) Displays.

Also provided according to embodiments of the invention, provided are automated chest compression devices that perform the methods described herein. Such chest compression devices or systems may include or be in electrical communication (including wireless communication) with the signal processor. When the signal processor is part of the chest compression device, it may also be referred to as the controller. The chest compression device may further include a compressor, which is the mechanical device that provides force to the individual's chest.

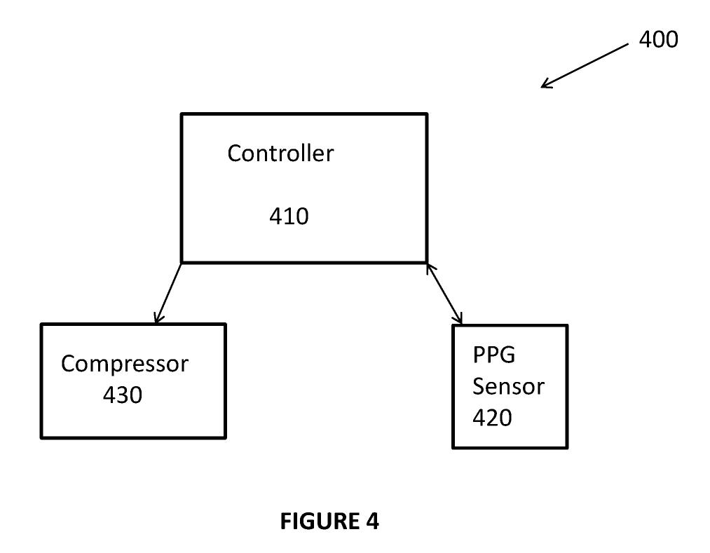

Also provided according to some embodiments of the invention are systems that include a nasal PPG sensor and the automated chest compression device. The PPG sensor and automated chest compression device include those described elsewhere within. FIG. 4 provides a simple graphic of an example of a chest compression system 400 that includes a controller 410 in communication with the PPG sensors 420 and the compressor(s) 430.

In some embodiments, the controller 410 receives and processes PPG signals from the PPG sensor 420 in the manner described above to determine a waveform parameter. For example, if the waveform parameter meets preselected criteria, the controller may direct the compressor(s) 430 to maintain, increase or decrease the depth of at least one subsequent chest compression based on the waveform parameter. In some cases, the controller 410 may direct the compressor 430 to maintain, increase or decrease a duration at least one subsequent chest compression based on the waveform parameter. The manner of doing so is described above (see, e.g., FIG. 2 and accompanying text), whereby the chest compressions are performed by the compressor 430, and the compressor 430 is instructed to maintain, increase or decrease the at least one of depth, duration and frequency of chest compressions by the controller.

### Return of Spontaneous Circulation

In addition to the methods, devices, and systems described above, the PPG signals from the nose may be used to determine whether the individual has a return of spontaneous circulation (ROSC). In some embodiments, this may be determined by assessing whether the pulsatile signal (either raw or the AC component) has returned in the absence of the chest compressions. Other physiological sensors/parameters, as described above, may also be used determine whether ROSC has occurred.

After ROSC, the amplitude or other waveform parameter may be monitored to determine whether adequate blood flow has been restored to the brain. The PPG sensor at the nose, as a surrogate for cerebral blood flow, provides this information. Even if electrical activity has been returned to the heart, the blood flow to the brain may still not be adequate or optimal. The PPG signal at the nose may provide information regarding whether CPR should continue beyond ROSC in order to supplement blood flow to the brain. Furthermore, by using the PPG signal at the nose, the chest compressions may be synchronized with the spontaneous heart beat/blood flow to provide increased (augmented) cerebral blood flow.

### EXAMPLE 1

In a prophetic example, a nasal alar sensor may be placed on a patient in cardiac arrest without stopping chest compressions. If chest compressions are effective, a PPG signal should be seen on a monitor displaying the signal. This could be a proprietary “CPR” monitor that in addition to displaying oxygen saturation would display the PPG waveform which would be valuable for determining the efficacy of chest compressions and the quality of cardiac output (especially cerebral blood flow) once the heart is restarted. The operator can vary the depth of chest compressions to see if there is an improvement in the PPG amplitude at a different depth of compression or if the present depth is optimal Likewise, if the PPG signal is “flat line” or is present but relatively small the depth of compressions can be varied to see if a larger PPG waveform can be produced with deeper compressions or compressions at a different rate. The PPG signal should then increase significantly with Return of Spontaneous Circulation (ROSC) since chest compressions only provide a small blood flow compared to normal cardiac output. The effectiveness of drug administration can also be monitored with the nasal sensor. Administration of epinephrine or other vasoactive drugs should improve the PPG amplitude as blood flow is preferentially redirected to the cerebral (and nasal) and coronary circulation.

### EXAMPLE 2

Monitoring from the nasal ala or septum can also be used in the research setting to see if other combinations of rate and depth of compression are superior to the present ACLS recommendations (which are largely inferred from animal studies). Using nasal sensors does not interfere with chest compression, and thus data can be collected continuously and defibrillation success rates can be analyzed post hoc in the context of the adequacy of the PPG signal.

In the specification, there have been disclosed embodiments of the invention and, although specific terms are employed, they are used in a generic and descriptive sense only and not for purposes of limitation, the scope of the invention being set forth in the following claims.

We claim:

1. A method for improving the effectiveness of chest compressions as part of cardiopulmonary resuscitation (CPR) comprising
(a) monitoring PPG signals from a PPG sensor secured to a nose of an individual during at least one chest compression; and
(b) increasing, decreasing or maintaining at least one of a depth, duration and frequency of at least one subsequent chest compression based on a waveform parameter of the PPG signals.

2. The method of claim 1, wherein the waveform parameter comprises an amplitude of the PPG signal, an area under the curve of the PPG signal, or both.

3. The method of claim 1, wherein if the waveform parameter of the PPG signals meets a preselected criteria, the depth of the chest compression is increased.

4. The method of claim 1, wherein if the waveform parameter of the PPG signals meets a preselected criteria, the duration of the chest compression is increased.

5. The method of claim 1, wherein PPG signals from the PPG sensor during two or more chest compressions are monitored; and the at least one of the depth, duration and frequency of the at least one additional chest compression is increased or decreased based on the PPG signals during the two or more chest compressions.

6. The method of claim 1, wherein the PPG sensor is secured to a nasal alar of the individual.

7. The method of claim 1, wherein the PPG signals are separated into at least one of an AC component signal and a DC component signal, and the at least one of the depth, duration and frequency of the at least one additional chest compression is maintained, increased or decreased based on a waveform parameter of the AC component signal, the DC component signal, or both.

8. The method of claim 1, further comprising iterating steps (a) and (b) to optimize the waveform parameter of the PPG signals.

9. The method of claim 1, further comprising using the PPG signals to determine at least one of blood oxygen saturation and a respiratory parameter.

10. An automated chest compression device comprising
(a) a compressor that performs chest compressions on an individual;
(b) a controller that activates the compressor to perform chest compressions on the individual,
wherein the controller receives and processes PPG signals from a PPG sensor secured to the nose; and
wherein the controller calculates a waveform parameter of the PPG signals during a chest compression and directs the compressor to maintain, increase or decrease at least one of a depth, duration and frequency of at least one subsequent chest compression based on the waveform parameter.

11. The automated chest compression device of claim 10, wherein the waveform parameter comprises an amplitude of the PPG signals.

12. The automated chest compression device of claim 10, wherein the waveform parameter comprises an area under a curve of the PPG signals.

13. The automated chest compression device of claim 10, wherein the controller is further configured to determine at least one of a blood oxygen saturation and a respiratory parameter from the PPG signal.

14. The automated chest compression device of claim 10, wherein the controller directs the compressor to increase or decrease the at least one of the depth, duration and frequency of the at least one additional chest compression based on the waveform parameters obtained during two or more previous chest compressions.

15. The automated chest compression device of claim 10, wherein the PPG signals are separated into at least one of an AC component signal and a DC component signal, and at least one of the depth, duration and frequency of the at least one additional chest compression is increased or decreased based on a waveform parameter of the AC component signal, the DC component signal, or both.

16. The method of claim 1, further comprising evaluating the PPG signals to determine when return of spontaneous circulation occurs.
